(19) United States
(12) Patent Application Publication
(10) Pub. No.: US 2010/0210510 A1
(43) Pub. Date: Aug. 19, 2010

(54) ORAL LACTOFERRIN IN THE TREATMENT OF SEPSIS

(75) Inventors: ATUL VARADHACHARY, Houston, TX (US); Karel Petrak, Houston, TX (US)

Correspondence Address: FULBRIGHT & JAWORSKI, LLP, 1301 MCKINNEY, SUITE 5100, HOUSTON, TX, 77010-3095, US

(73) Assignee: AGENNIX INC, Houston, TX (US)

(21) Appl. No.: 12/702,216

(22) Filed: Feb. 8, 2010

Related U.S. Application Data
(63) Continuation of application No. 10/728,521, filed on Dec. 5, 2003, now abandoned.
(60) Provisional application No. 60/431,393, filed on Dec. 6, 2002.
(60) Provisional application No. 60/498,327, filed on Aug. 27, 2003.

(51) Int. Cl. A61K 38/40; A61P 7/00; A61P 31/04

(52) U.S. Cl. 514/8

(57) ABSTRACT

The present invention relates to methods of treating prophylactically or therapeutically bacteremia, sepsis, septic shock or related conditions such as ARDS by administering orally a composition of lactoferrin alone or in combination with standard therapies or metal chelators to prevent or treat the consequences of bacterially induced systemic inflammatory response syndrome. In particular it is claimed that the therapeutic use of recombinant human lactoferrin alone or in combination with metal chelators or other therapeutic interventions decreases the mortality due to bacteremia, sepsis, septic shock or related conditions such as ARDS.

### CROSS-REFERENCE TO RELATED APPLICATIONS

This application is a continuation under 35 U.S.C. 120 of U.S. patent application Ser. No. 10/728,521, filed 5 Dec. 2003, which claims under 35 U.S.C. 119(e) the benefit of U.S. Provisional Application Nos. 60/431,393 filed Dec. 6, 2002 and 60/498,327 filed Aug. 27, 2003. Each of the priority applications are incorporated herein by reference.

### TECHNICAL FIELD

The present invention relates to methods of treating bacteremia, sepsis, septic shock or related conditions such as Acute Respiratory Distress Syndrome (ARDS) by administering orally a composition of lactoferrin (LF) alone or in combination with standard therapies or metal chelators, such as EDTA (ethylenediaminetetraacetic acid). More particularly, the present invention relates to methods of treating prophylactically or therapeutically endotoxemia, gram-negative and gram-positive bacteremia, sepsis, septic shock or related conditions such as ARDS by administering orally a composition of lactoferrin alone, or in combination with a metal chelator or in combination with standard therapies.

### BACKGROUND OF THE INVENTION

Sepsis is defined as the Systemic Inflammatory Response Syndrome (SIRS) to an infective process. Sepsis is a result of a bacterial infection that can originate anywhere in the body. Common sites are the genitourinary tract, the liver or biliary tract, the gastrointestinal tract, and the lungs. Less common sites are intravenous lines, surgical wounds, decubitus ulcers and bedsores. The infection is usually confirmed by a positive blood culture. The infection can lead to a shock, called septic shock. Septic shock is more often caused by hospital-acquired gram-negative bacilli and usually occurs in immunocompromised patients and those with chronic diseases. In about ⅓ of patients, however, it is caused by gram-positive cocci and by Candida organisms. The diagnosis of sepsis is based on the presence of at least two out of the following four criteria: tachycardia (heart rate>90 bpm), hyperventilation (respiratory frequency>20/min or pCO₂exp<35 mm Hg), fever (>38.3° C.) or hypothermia (<36° C.) and leukocytosis (>12,000/4) or leukopenia (<4,000/μL).

There are about 750,000 cases of sepsis in the U.S.A. every year, at least 225,000 of which are fatal. Only one drug has been approved for sepsis so far—a recombinant human activated protein C that exhibits antithrombotic, anti-inflammatory and profibrinolytic properties.

The pathogenesis of septic shock resulting from bacteremia and sepsis (SIRS) is not completely understood. The bacterial toxins generated by the infecting organisms trigger complex immunologic reactions. A large number of mediators including tumour necrosis factor, leukotriens, lipoxygenase, histamine, bradykinin, serotonin, and interleukin-2 have been implicated in addition to endotoxin (the lipid fraction of the lipopolysaccharides released from the cell wall of gram-negative enteric bacilli). Initially, vasodilation of arteries and arterioles occurs, decreasing peripheral arterial resistance with normal or increased cardiac output even though the ejection fraction may be decreased when heart rate increases. Later, cardiac output may decrease and peripheral resistance may increase. Despite increased cardiac output, blood flow to the capillary exchange vessels is impaired causing eventually failure of one or more of the visceral organs.

In experimental animals, for example in mice injected with endotoxin, endotoxemia and endotoxin-induced death is accompanied by oxidative burst and overproduction of inflammatory mediators. Intraperitoneally administered lactoferrin has been described to influence the outcome of endotoxemia, primarily through binding to the bacterial endotoxins (Kruzel M L et al., 2002). Other effects of parenteral lactoferrin have also been described for example, intraperitoneal administration of lactoferrin 1 hour before lipopolysaccharide (LPS) challenge resulted in an inhibition of several mediators, namely TNF-α by 82%, IL-6 by 43%, IL-10 by 47% at 2 hours following LPS injection, and reduction in nitric oxide (NO) (by 80%) at 6 hours post-shock. Prophylactic i.p. administration of lactoferrin at 18 hours prior to LPS injection resulted in similar decreases in TNF-α (95%) and in NO (62%). Similarly, when lactoferrin was administered i.p. as a therapeutic post-induction of endotoxic shock, significant reductions were apparent in TNF-α and NO in serum.

It has been reported in the literature that oral lactoferrin is not absorbed systemically through the mature gut to any significant degree (Heyman M et al., 1992; Fransson G B et al., 1983; and Holloway N M et al., 2002) and the literature also assumes that a large part of lactoferrin's role is related to the binding of systemically circulating endotoxins. For example, a GLP pharmacokinetic study was conducted in the Rhesus monkey to determine the oral availability of rhLF. Standard dosing volume of 4 mL/kg was administered by oral gavage. A comparison was made to the pharmacokinetics of i.v.-administered rhLF. The oral dose of rhLF was 1000 mg/kg. Following this oral dose, the plasma concentrations of rhLF were not significantly higher than the pre-dose, endogenous lactoferrin values. The calculated absolute bioavailability of hLF was less than 0.5% (Fransson G B et al., 1983; Heymen M et al., 1992).

Lactoferrin is a single chain metal binding glycoprotein. Many cells types, such as monocytes, macrophages, lymphocytes, and brush-border cells, are known to have lactoferrin receptors. In addition to lactoferrin being an essential growth factor for both B and T lymphocytes, lactoferrin has a wide array of functions related to host primary defense mechanisms. For example, lactoferrin has been reported to activate natural killer (NK) cells, induce colony-stimulating activity, activate polymorphonuclear neutrophils (PMN), regulate granulopoeisis, enhance antibody-dependent cell cytotoxicity, stimulate lymphokine-activated killer (LAK) cell activity, and potentiate macrophage toxicity.

Recombinant human lactoferrin has previously been described as being purified after expression in a variety of prokaryotic and eukaryotic organisms including aspergillus (U.S. Pat. No. 6,080,559), cattle (U.S. Pat. No. 5,919,913), rice, corn, Sacharomcyes (U.S. Pat. No. 6,228,614) and Pichia pastoris (U.S. Pat. Nos. 6,455,687, 6,277,817, 6,066,469). Also described are expression systems for the expression of full-length human lactoferrins (e.g., U.S. Pat. No. 6,100,054). In all cases, part of the teaching is expression of the full-length cDNA and purification of the intact protein whose N-terminal, after processing of the leader peptide, is the amino acid glycine. Nuijens et al. (U.S. Pat. No. 6,333,311) separately describe variants of human lactoferrin but their focus is limited to deletion or substitution of arginine residues found in the N-terminal domain of lactoferrin.

The present invention is the first to use an oral lactoferrin composition as a treatment or prophylaxis for systemic bacteremia, sepsis, septic shock or related conditions. Further, the present invention is the first to use lactoferrin in combination with a metal chelator to treat systemic bacteremia, sepsis, septic shock or related conditions. Yet further, the present invention is the first to use lactoferrin in combination with existing therapy to treat systemic bacteremia, sepsis, septic shock or related conditions.

### BRIEF SUMMARY OF THE INVENTION

The present invention is directed to a method for treating prophylatically or therapeutically bacteremia, sepsis, septic shock or related conditions such as multiple organ failure and acute respiratory distress syndrome (ARDS). The method of treatment involves oral administration of a lactoferrin composition alone or in combination with a metal chelator.

The lactoferrin composition, which is dispersed in a pharmaceutically acceptable carrier, comprises lactoferrin or an N-terminal lactoferrin variant in which at least the N-terminal glycine residue is truncated or substituted. The lactoferrin is mammalian lactoferrin, more particularly, the lactoferrin is human or bovine. Yet further, the lactoferrin is recombinant lactoferrin. N-terminal lactoferrin variants include variants that at least lack the N-terminal glycine residue or contain a substitution at the N-terminal glycine residue. The substitution can comprise substituting a natural or artificial amino acid residue for the N-terminal glycine residue. For example, the substitution can comprise substituting a positive amino acid residue or a negative amino acid residue for the N-terminal glycine residue or substituting a neutral amino acid residue other than glycine for the N-terminal glycine residue. Other N-terminal lactoferrin variants include lactoferrin lacking one or more N-terminal residues or having one or more substitutions in the N-terminal. In specific embodiments, the N-terminal lactoferrin variant comprises at least 1% of the lactoferrin composition, at least 5% of the lactoferrin composition, at least 10% of the lactoferrin composition, at least 25% of the lactoferrin composition, at least 50% of the lactoferrin composition or any range in between.

The amount of the lactoferrin that is orally administered is about 1 mg to about 100 g per day, more preferably, the amount is about 10 mg to about 10 g per day. More particularly, the composition is a solution, capsule or a tablet having a lactoferrin concentration of about 0.1% to about 100%.

In further embodiments, a metal chelator dispersed in a pharmaceutically acceptable carrier can also be administered with the lactoferrin composition. Preferred metal chelator include, but are not limited to ethylenediaminetetraacetic acid (EDTA) or [ethylenebis(oxyethylenenitrilo)]tetraacetic acid (EGTA). More preferably, the metal chelator is EDTA. The amount of EDTA that is administered is about 0.01 μg to about 20 g per day. The ratio of EDTA to lactoferrin in the composition that is administered is from 1:10,000 to about 2:1.

An embodiment of the present invention is a method of treating bacteremia comprising the step of administering orally to a subject a lactoferrin composition in an effective amount to provide an improvement in the bacteremia of the subject. The improvement is attenuating sepsis, attenuating septic shock, attenuating organ failure, decreasing morbidity, and/or a decreasing mortality. More specifically, oral administration is via a nasogastric tube. Yet further, the lactoferrin composition can be administered in combination with an antibiotic.

For oral administration, an antacid in combination with the lactoferrin composition can be administered. The lactoferrin can be formulated in a delayed release formulation. Still further, the lactoferrin composition can be formulated wherein release occurs in the small intestine or in the large intestine. The composition that is administered is a liquid formulation, a solid formulation with an enteric coating or a solid formulation without an enteric coating.

Another embodiment of the present invention is a method of treating bacteremia comprising the step of supplementing the mucosal immune system in a subject by administering orally to the subject an amount of a lactoferrin composition to increase the amount of lactoferrin in the gastrointestinal tract. More specifically, oral administration is via a nasogastric tube.

Still further, another embodiment is a method of enhancing a mucosal immune response in the gastrointestinal tract in a subject comprising the step of administering orally to said subject a lactoferrin composition. The lactoferrin stimulates interleukin-18 in the gastrointestinal tract. Interleukin-18 stimulates the production or activity of immune cells. The lactoferrin reduces the production or activity of pro-inflammatory cytokines.

Another embodiment is a method of preventing bacteremia in a subject at risk of developing bacteremia comprising the step of administering orally to said subject a composition having lactoferrin and a metal chelator in an effective amount to prevent or attenuate the bacteremia in said subject. More specifically, oral administration is via a nasogastric tube. A subject at risk for developing bacteremia can be an immunocompromised subject.

A specific embodiment is a method of decreasing mortality of a subject having bacteremia comprising the step of administering orally to said subject a lactoferrin composition in an effective amount to attenuate the bacteremia to decrease mortality of said subject.

Another embodiment is a method of treating a septic condition in a subject comprising the step of administering orally to said subject a lactoferrin composition in an effective amount to provide an improvement in the septic condition of said subject. The improvement is decreasing the levels of circulating bacteria, attenuating septic shock, attenuating organ failure, decreasing morbidity, or decreasing mortality.

A further embodiment is a method of decreasing mortality of a subject having sepsis comprising the step of administering orally to said subject a lactoferrin composition in an effective amount to attenuate sepsis to decrease mortality of said subject. The composition reduces the levels of circulating cytokines, for example, the cytokines are selected from the group consisting of IL-4, IL-6, TNF-α and IL-10. Still further, the method comprises administering the lactoferrin composition in combination with an approved therapy for sepsis, for example Drotrecogin alfa (activated) or Xigris®.

Another embodiment is a method of decreasing mortality of a subject having Acute Lung Injury (ALI) or Acute Respiratory Distress Syndrome (ARDS) comprising the step of administering orally to said subject a lactoferrin composition in an effective amount to attenuate ALI or ARDS to decrease mortality of said subject. Still further, the method comprises administering the lactoferrin composition in combination with a standard therapy for ALI/ARDS, for example is low tidal volume ventilation or surfactant.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated that the conception and specific embodiment disclosed may be readily utilized as a basis for modifying or designing other structures for carrying out the same purposes of the present invention. It should also be realized that such equivalent constructions do not depart from the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing.

FIG. 1 compares the effect of oral and intravenous administration of rhLF, at different doses and dose regimens, on decreasing the mortality of mice in an LPS-induced endotoxemia model.

FIG. 2 shows the reduction of mortality and key cytokines in sepsis.

### DETAILED DESCRIPTION OF THE INVENTION

It is readily apparent to one skilled in the art that various embodiments and modifications can be made to the invention disclosed in this application without departing from the scope and spirit of the invention.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having”, “including” and “comprising” are interchangeable and one of skill in the art is cognizant that these terms are open ended terms.

The term “antimicrobial” as used herein is defined as a substance that inhibits the growth of microorganisms without damage to the host, for example antibiotics, anti-fungal and antiseptics.

The term “antibiotics” as used herein is defined as a substance that inhibits the growth of microorganisms without damage to the host. For example, the antibiotic may inhibit cell wall synthesis, protein synthesis, nucleic acid synthesis, or alter cell membrane function. Classes of antibiotics that can possibly be used include, but are not limited to, macrolides (e.g., erythromycin), penicillins (e.g., nafcillin), cephalosporins (e.g., cefazolin), carbepenems (e.g., imipenem, aztreonam), other beta-lactam antibiotics, beta-lactam inhibitors (e.g., sulbactam), oxalines (i.e. linezolid), aminoglycosides (e.g., gentamicin), chloramphenicol, sulfonamides (e.g., sulfamethoxazole), glycopeptides (e.g., vancomycin), quinolones (e.g., ciprofloxacin), tetracyclines (e.g., minocycline), fusidic acid, trimethoprim, metronidazole, clindamycin, mupirocin, rifamycins (e.g., rifampin), streptogramins (e.g., quinupristin and dalfopristin) lipoprotein (e.g., daptomycin), polyenes (e.g., amphotericin B), azoles (e.g., fluconazole), and echinocandins (e.g., caspofungin acetate). The term “morbidity” as used herein is the state or condition of being diseased. Yet further, morbidity can also refer to the ratio of incidence, for example the number of sick subjects or cases of diseases in relationship to a specific population.

The term “bacteremia” as used herein is defined as having a focus of bacterial infection or bacteria in the blood of the subject.

The term “chemokine” as used herein refers to small cytokines that are involved in the migration and activation of cells, for example phagocytic cells and lymphocytes. One of skill in the art realizes that chemokines play a central role in inflammatory and immune response processes.

The term “cytokine” as used herein refers to proteins that are made by cells that affect the behavior of other cells, for example stimulate or inhibit cell proliferation. For example, cytokines that are made by lymphocytes are often called lymphokines or interleukins. One of skill in the art realizes that the term cytokine is a generic term used in the literature to refer to proteins that are made by cells that can effect the behavior of other cells.

The term “effective amount” or “therapeutically effective amount” as used herein refers to an amount that results in an improvement or remediation of the symptoms of the disease or condition.

The term “endotoxin” as used herein refers to a bacterial toxin not freely liberated into the surrounding medium.

The term “endotoxemia” as used herein refers to the presence of endotoxins in the blood.

The term “gram-negative bacteria” or “gram-negative bacterium” as used herein is defined as bacteria which have been classified by the Gram stain as having a red stain. Gram-negative bacteria have thin walled cell membranes consisting of a single layer of peptidoglycan and an outer layer of lipopolysaccharide, lipoprotein, and phospholipid. Exemplary organisms include, but are not limited to, Enterobacteriacea consisting of Escherichia, Shigella, Edwardsiella, Salmonella, Citrobacter, Klebsiella, Enterobacter, Hafnia, Serratia, Proteus, Morganella, Providencia, Yersinia, Erwinia, Buttlauxella, Cedecea, Ewingella, Kluyvera, Tatumella and Rahnella. Other exemplary gram-negative organisms not in the family Enterobacteriacea include, but are not limited to, Pseudomonas aeruginosa, Stenotrophomonas maltophilia, Burkholderia, Cepacia, Gardenerella, Vaginalis, and Acinetobacter species.

The term “gram-positive bacteria” or “gram-positive bacterium” as used herein refers to bacteria, which have been classified using the Gram stain as having a blue stain. Gram-positive bacteria have a thick cell membrane consisting of multiple layers of peptidoglycan and an outside layer of teichoic acid. Exemplary organisms include, but are not limited to, Staphylococcus aureus, coagulase-negative staphylococci, streptococci, enterococci, corynebacteria, and Bacillus species.

The term “immunocompromised” as used herein is defined as a subject who is, at the time of pathogen exposure, has a pre-existing condition that reduces one or more mechanisms for normal defense against infection. The immunocompromised condition may be due to a defect or dysfunction of the immune system or to other factors that heighten susceptibility to infection, for example immunosuppressive agents. Although such a categorization allows a conceptual basis for evaluation, immunocompromised individuals with infection often do not fit completely into one group or the other. More than one defect in the body's defense mechanisms may be affected. For example, an immunocompromised state can result from indwelling central lines or other types of impairment due to intravenous drug abuse; or be caused by secondary malignancy, malnutrition, or having been infected with other infectious agents such as tuberculosis, influenza, Staphylococcus aureus or sexually transmitted diseases, e.g., syphilis or hepatitis.

The term “lactoferrin” or “LF” as used herein refers to native or recombinant lactoferrin. Native lactoferrin can be obtained by purification from mammalian milk or colostrum or from other natural sources. Recombinant lactoferrin (rLF) can be made by recombinant expression or direct production in genetically altered animals, plants, fungi, bacteria, or other prokaryotic or eukaryotic species, or through chemical synthesis.

The term “lactoferrin composition” as used herein refers to a composition having lactoferrin, a portion or part of lactoferrin, an N-terminal lactoferrin variant, or a combination thereof.

The term “mortality” as used herein is the state of being mortal or causing death. Yet further, mortality can also refer to the death rate or the ratio of number of deaths to a given population.

The term “morbidity” as used herein is the state of being diseased. Yet further, morbidity can also refer to the disease rate or the ratio of sick subjects or cases of disease in to a given population.

The term “metal chelator” as used herein refers to a compound which binds metal. Metal chelators that can be used in the present invention include the divalent metal chelators, for example, ethylenediaminetetraacetic acid (EDTA), [ethylenebis (oxyethylenenitrilo)]tetraacetic acid (EGTA), 1,2-bis(2-aminophenoxy)ethane-N,N,N′,N′-tetraacetic acid (BAPTA), hydroxyethylethylene diamine triacetic acid, (HEDTA) or salts thereof.

The term “N-terminal lactoferrin variant” as used herein refers to lactoferrin wherein at least the N-terminal glycine has been truncated and/or substituted. N-terminal lactoferrin variants also include, but are not limited to deletion and/or substitution of one or more N-terminal amino acid residues, for example 1, 2, 3, 4, 5, 6, 7, 8, 9, 10, 11, 12, 13, 14, 15, or 16 N-terminal amino acid residues, etc. Thus, N-terminal lactoferrin variants comprise at least deletions or truncations and/or substitutions of 1 to 16 N-terminal amino acid residues. The deletion and/or substitution of at least the N-terminal glycine of lactoferrin mediates the same biological effects as full-length lactoferrin and/or may enhance lactoferrin's biological activity, for example by stimulating the production of various cytokines (e.g., IL-18, MIP-3α, GM-CSF or IFN-γ) by inhibiting various cytokines, (e.g., IL-2, IL-4, IL-5, IL-6, IL-10, and TNF-α) by attenuating sepsis, attenuating septic shock, attenuating organ failure, decreasing morbidity, and/or decreasing mortality.

The term “oral administration” as used herein includes oral, buccal, enteral, rectal or intragastric administration.

The term “pharmaceutically acceptable carrier” as used herein includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

The term “preventing” as used herein refers to minimizing, reducing or suppressing the risk of developing a disease state or parameters relating to the disease state or progression or other abnormal or deleterious conditions.

The term “sepsis” as used herein is defined as a Systemic Inflammatory Response Syndrome to an infective process in which severe derangement of the host immune system fails to prevent extensive ‘spill over’ of inflammatory mediators from a local infection focus into the systemic circulation.

The term “septic shock” as used herein is a consequence of sepsis in which the systemic inflammatory response leads to the failure of vital organs' function (for example of the lungs as in ARDS).

The term “subject” as used herein, is taken to mean any mammalian subject to which a lactoferrin composition is orally administered according to the methods described herein. A skilled artisan realizes that a mammalian subject, includes, but is not limited to humans, monkeys, horses, pigs, cows, dogs, cats, rats and mice. In a specific embodiment, the methods of the present invention are employed to treat a human subject. In further embodiments, the subject is at risk of developing bacteremia or sepsis. Thus, the subject may or may not be cognizant of their disease state or potential disease state and may or may not be aware that they are need of treatment (therapeutic treatment or prophylactic treatment).

The term “treating” and “treatment” as used herein refers to administering to a subject a therapeutically effective amount of a recombinant human lactoferrin composition so that the subject has an improvement in the disease. The improvement is any improvement or remediation of the symptoms associated with bacteremia, sepsis, septic shock or their consequences. The improvement is an observable or measurable improvement, for example, decreased levels of circulating bacteria, decrease in mortality, decrease in morbidity, attenuating the development of organ failure, decreasing days of hospitalization, decreasing or eliminating days of intensive care such as in an intensive care unit, or decreasing or eliminating the use of supportive care such as a mechanical ventilator or PaO₂/FiO₂ ratio. Thus, one of skill in the art realizes that a treatment may improve the disease condition, but may not be a complete cure for the disease.

### A. LACTOFERRIN

The lactoferrin used according to the present invention can be obtained through isolation and purification from natural sources, for example, but not limited to mammalian milk. The lactoferrin is preferably mammalian lactoferrin, such as bovine or human lactoferrin. In preferred embodiments, the lactoferrin is produced recombinantly using genetic engineering techniques well known and used in the art, such as recombinant expression or direct production in genetically altered animals, plants or eukaryotes, or chemical synthesis. See, e.g., U.S. Pat. Nos. 5,571,896; 5,571,697 and 5,571,691, which are herein incorporated by reference.

In certain aspects, the present invention provides lactoferrin variants having enhanced biological activities over natural LF and or rLF, e.g., the ability to stimulate and/or inhibit cytokines or chemokines. In particular, the invention provides variants of lactoferrin from which at least the N-terminal glycine residue has been substituted and/or truncated. The N-terminal lactoferrin variants may occur naturally or may be modified by the substitution or deletion of one or more amino acids.

The deletional variants can be produced by proteolysis of lactoferrin and/or expression of a polynucleotide encoding a truncated lactoferrin as described in U.S. Pat. No. 6,333,311, which is incorporated herein by reference.

Substitutional variants or replacement variants typically contain the exchange of one amino acid for another at one or more sites within the protein. Substitutions can be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics (Kyte and Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, e.g., still obtain a biological functionally equivalent protein. In making such changes, the substitution of amino acids whose hydropathic indices are within ±2 is preferred, those that are within ±1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine −0.5); cysteine (−1.0); methionine (−1.3); valine (−1.5); leucine (−1.8); isoleucine (−1.8); tyrosine (−2.3); phenylalanine (−2.5); tryptophan (−3.4).

Still further, it is understood that an amino acid can be substituted for another having a similar hydrophilicity value and still obtains a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

Thus, in the present invention, substitutional variants or replacement can be produced using standard mutagenesis techniques, for example, site-directed mutagenesis as disclosed in U.S. Pat. Nos. 5,220,007; 5,284,760; 5,354,670; 5,366,878; 5,389,514; 5,635,377; 5,789,166, and 6,333,311, which are incorporated herein by reference. It is envisioned that at least the N-terminal glycine amino acid residue can be replaced or substituted with any of the twenty natural occurring amino acids, for example a positively charged amino acid (arginine, lysine, or histidine), a neutral amino acid (alanine, asparagine, cysteine, glutamine, glycine, isoleucine, leucine, methionine, phenylaline, proline, serine, threonine, tryptophan, tyrosine, valine) and/or a negatively charged amino acid (aspartic acid or glutamic acid). Still further, it is contemplated that any amino acid residue within the range of N1 to N16 can be replaced or substituted. It is envisioned that at least up to 16 of the N-terminal amino acids residues can be replaced or substituted as long as the protein retains it biological and/or functional activity, which is stimulating the production of various cytokines (e.g., IL-18, MIP-3α, GM-CSF or IFN-γ), by inhibiting various cytokines, (e.g., IL-2, IL-4, IL-5, IL-6, IL-10, or TNF-α), by attenuating sepsis, attenuating septic shock, attenuating organ failure, decreasing morbidity, and/or decreasing mortality. Thus, the N-terminal lactoferrin variants of the present invention are considered functional equivalents of lactoferrin.

In terms of functional equivalents, it is well understood by the skilled artisan that, inherent in the definition of a “biologically functional equivalent” protein is the concept that there is a limit to the number of changes that may be made within a defined portion of the molecule while retaining a molecule with an acceptable level of equivalent biological activity and/or enhancing the biological activity of the lactoferrin molecule. Biologically functional equivalents are thus defined herein as those proteins in which selected amino acids (or codons) may be substituted. Functional activity is defined as the ability of lactoferrin to stimulate or inhibit various cytokines or chemokines and/or attenuate sepsis, attenuate septic shock, attenuate organ failure, decrease morbidity, and/or decrease mortality.

Still further, the N-terminal amino acid residues can be substituted with a modified and/or unusual amino acids. A table of exemplary, but not limiting, modified and/or unusual amino acids is provided herein below.

TABLE 1
Modified and/or Unusual Amino Acids

| Abbr. | Amino Acid | Abbr. | Amino Acid |
|---|---|---|---|
| Aad | 2-Aminoadipic acid | EtAsn | N-Ethylasparagine |
| BAad | 3-Aminoadipic acid | Hyl | Hydroxylysine |
| BAla | beta-alanine, beta-Amino-propionic acid | AHyl | allo-Hydroxylysine |
| Abu | 2-Aminobutyric acid | 3Hyp | 3-Hydroxyproline |
| 4Abu | 4-Aminobutyric acid, piperidinic acid | 4Hyp | 4-Hydroxyproline |
| Acp | 6-Aminocaproic acid | Ide | Isodesmosine |
| Ahe | 2-Aminoheptanoic acid | Aile | allo-Isoleucine |
| Aib | 2-Aminoisobutyric acid | MeGly | N-Methylglycine, sarcosine |
| BAib | 3-Aminoisobutyric acid | MeIle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic acid | Orn | Ornithine |
| EtGly | N-Ethylglycine | | |

The presence and the relative proportion of an N-terminal lactoferrin variants (deletions and/or substitutions) in a preparation of lactoferrin (lactoferrin composition) may be done by determination of the N-terminal amino acid sequence by the process of Edman degradation using standard methods. A relative proportion of N-terminal lactoferrin variant comprises at least 1% of the lactoferrin composition, at least 5% of the lactoferrin composition, at least 10% of the lactoferrin composition, at least 25% of the lactoferrin composition, at least 50% of the lactoferrin composition or any range in between.

In this method, the protein is reacted with phenylisothiocyanate (PITC), which reacts with the amino acid residue at the amino terminus under basic conditions to form a phenylthiocarbamyl derivative (PTC-protein). Trifluoroacetic acid then cleaves off the first amino acid as its anilinothialinone derivative (ATZ-amino acid) and leaves the new amino terminus for the next degradation cycle.

The percentage of N-terminal lactoferrin variant may also be done more precisely by using a Dansylation reaction. Briefly, protein is dansylated using Dansyl chloride reacted with the protein in alkaline conditions (pH 10). Following the Dansylation, the reaction mixtures are dried to pellets, then completely hydrolyzed in 6N HCl. The proportion of N-terminal amino acids are identified by RP HPLC using an in-line fluorimeter in comparison with standards made up of known dansylated amino acids.

### B. PHARMACEUTICAL COMPOSITIONS

The present invention is drawn to a composition comprising a lactoferrin composition that is dispersed in a pharmaceutical carrier. The lactoferrin that is contained in the composition of the present invention comprises lactoferrin or an N-terminal lactoferrin variant in which at least the N-1 terminal glycine residue is truncated or substituted. More specifically, the N-terminal lactoferrin variant comprises at least 1% of the composition, at least 5% of the composition, at least 10% of the composition, at least 25% of the composition, at least 50% of the composition or any range in between.

Yet further, the composition comprises lactoferrin in combination with a metal chelator dispersed in a pharmaceutical carrier. Thus, the present invention is drawn to a lactoferrin composition with or without a metal chelator that is dispersed in a pharmaceutical carrier. One of skill in the art understands that both compositions (e.g., lactoferrin alone or lactoferrin in combination with a metal chelator) are within the scope of the present invention and can be used interchangeably depending upon the type of response that is desired. It is envisioned that the addition of a metal chelator to the lactoferrin composition enhances the sequestering of metal ions and thus strengthens the immune system or enhances the effect of lactoferrin.

Metal chelators that can be used in combination with lactoferrin, include the divalent metal chelators, for example, ethylenediaminetetraacetic acid (EDTA), [ethylenebis(oxyethylenenitrilo)]tetraacetic acid (EGTA), 1,2-bis(2-aminophenoxy)ethane-N,N,N′,N′-tetraacetic acid (BAPTA), hydroxyethlene triamine diacetic acid, (HEDTA) or any salts thereof. More preferably, EDTA is used in combination with lactoferrin.

Further in accordance with the present invention, the composition of the present invention suitable for administration is provided in a pharmaceutically acceptable carrier with or without an inert diluent. The carrier should be assimilable and includes liquid, semi-solid, e.g., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof.

In accordance with the present invention, the composition is combined with the carrier in any convenient and practical manner, e.g., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, e.g., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc., proteolytic enzyme inhibitors, and the like. Yet further, it is envisioned that divalent metal chelators, for example EDTA, can also be used to stabilize the composition of the present invention. More preferably, for an orally administered composition, the stabilizer can also include antagonists to the secretion of stomach acids.

The composition for oral administration which is combined with a semi-solid or solid carrier can be further formulated into hard or soft shell gelatin capsules, tablets, or pills. More preferably, gelatin capsules, tablets, or pills are enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the lactoferrin composition to be released and absorbed by specialized cells, e.g., epithelial enterocytes and Peyer's patch M cells.

In another embodiment, a powdered composition is combined with a liquid carrier such as, e.g., water or a saline solution, with or without a stabilizing agent.

The amount of lactoferrin in the present invention may vary from about 1 g to about 100 g of lactoferrin. In preferred embodiments, the composition of the present invention comprises a lactoferrin concentration of about 0.0001% to about 30%. More preferably, lactoferrin is orally administered in the range of 10 mg to 10 g or lactoferrin. The lactoferrin may comprise lactoferrin or an N-terminal lactoferrin variant in which at least the N-1 terminal glycine residue is truncated and/or substituted.

More preferably, the composition of the present invention also contains metal chelators, for example, but not limited to ethylenediaminetetraacetic acid (EDTA), [ethylenebis(oxyethylenenitrilo)]tetraacetic acid (EGTA), 1,2-bis(2-aminophenoxy)ethane-N,N,N′,N′-tetraacetic acid (BAPTA), hydroxyethlene triamine diacetic acid, (HEDTA) or salts thereof. The amount of the metal chelator in the composition may vary from about 1 ng to about 20 g. A preferred metal chelator is EDTA.

Upon formulation, solutions are administered in a manner compatible with the dosage formulation and in such amount as is therapeutically effective to result in an improvement or remediation of the symptoms. The formulations are easily administered in a variety of dosage forms such as ingestible solutions, drug-release capsules and the like. Some variation in dosage can occur depending on the condition of the subject being treated. The person responsible for administration can, in any event, determine the appropriate dose for the individual subject.

### C. TREATMENT AND/OR PROPHYLAXIS

In accordance with the present invention, the composition provided in any of the above-described pharmaceutical carriers is orally administered to a subject suspected of or having bacteremia, sepsis, septic shock or sequelae. These conditions could be caused by gram-negative, gram-positive bacteria or other infectious agents such as Candida in any foci of the body and are at a risk of developing into or have developed into a systemic inflammatory response syndrome. One skilled in the art can determine the therapeutically and/or prophylatically effective amount of the composition to be administered to a subject based upon several considerations, such as local effects, pharmacodynamics, absorption, metabolism, method of delivery, age, weight, disease severity and response to the therapy. Oral administration of the composition includes oral, buccal, enteral, rectal or intragastric administration.

Bacteremia can be caused by gram-negative or gram-positive bacteria. Gram-negative bacteria have thin walled cell membranes consisting of a single layer of peptidoglycan and an outer layer of lipopolysacchacide, lipoprotein, and phospholipid. Exemplary gram-negative organisms include, but are not limited to, Enterobacteriacea consisting of Escherichia, Shigella, Edwardsiella, Salmonella, Citrobacter, Klebsiella, Enterobacter, Hafnia, Serratia, Proteus, Morganella, Providencia, Yersinia, Erwinia, Buttlauxella, Cedecea, Ewingella, Kluyvera, Tatumella and Rahnella. Other exemplary gram-negative organisms not in the family Enterobacteriacea include, but are not limited to, Pseudomonas aeruginosa, Stenotrophomonas maltophilia, Burkholderia, Cepacia, Gardenerella, Vaginalis, and Acinetobacter species. Gram-positive bacteria have a thick cell membrane consisting of multiple layers of peptidoglycan and an outside layer of teichoic acid. Exemplary gram-positive organisms include, but are not limited to, Staphylococcus aureus, coagulase-negative staphylococci, streptococci, enterococci, corynebacteria, and Bacillus species.

For example, bacteremia may be caused by surgical manipulation of infected oral tissues or routine dental manipulations; catheterization of an infected lower urinary tract; incision and drainage of an abscess; and colonization of indwelling devices, especially IV and intracardiac catheters, urethral catheters, and ostomy devices and tubes. The primary site of infection is usually in the lungs, in the GU or GI tract, or in soft tissues including the skin in patients with decubitus ulcer. In chronically ill and immunocompromised subjects, gram-negative bacteremia occurs more commonly, than in a healthy subject. Additionally, these immunocompromised subjects may develop bloodstream infections caused by aerobic bacilli, anaerobes, and fungi.

Predisposing factors for septic shock include diabetes mellitus; cirrhosis; leukopenic states, especially those associated with underlying neoplasms or treatment with cytotoxic agents; antecedent infection in the urinary, biliary, or GI tracts; invasive devices, including catheters, drainage tubes, and other foreign materials; and prior treatment with antibiotics, corticosteroids, or ventilatory devices. Septic shock occurs more often in newborns, subjects>35 yr, pregnant women, and those seriously immunocompromised by underlying diseases or iatrogenic complications of treatment.

In further embodiments, the composition is administered in conjunction with an antacid. Thus, an antacid is administered prior or substantially simultaneously with or after oral administration of the composition. The administration of an antacid just prior or immediately following the administration of the composition may help to reduce the degree of inactivation of the lactoferrin in the digestive tract. Examples of appropriate antacids include, but are not limited to, sodium bicarbonate, magnesium oxide, magnesium hydroxide, calcium carbonate, magnesium trisilicate, magnesium carbonate and aluminum hydroxide gel.

According to the invention, the above-described method is used for the prophylaxis of bacteremia, sepsis, septic shock, related conditions or their consequences. In specific embodiments, the disorder is characterized by a risk of endotoxemia resulting from the use of antibiotic and the subsequent release of endotoxin, as well as positively identified bacteremia.

Yet further, another embodiment is a method of preventing bacteremia in a subject at risk for developing bacteremia comprising the step of administering to the subject a lactoferrin composition in an amount sufficient to result in prophylaxis of bacteremia in the subject. It is envisioned that the lactoferrin composition not only possess therapeutic benefits for those subjects suffering from bacteremia, but also possess prophylactic properties for those subjects at risk for developing bacteremia, sepsis, septic shock and related conditions. A subject at risk may or may not be cognizant of their disease state or potential disease state and may or may not be aware that they are need of treatment.

A person at risk for developing bacteremia, sepsis, septic shock and/or related conditions is a person that is considered to be immunocompromised and/or chronically ill. The immunocompromised subject who is, at the time of bacterial exposure, has a pre-existing condition that reduces one or more mechanisms for normal defense against infection. The immunocompromised condition may be due to a defect or dysfunction of the immune system or to other factors that heighten susceptibility to infection, for example immunosuppressive agents.

Prophylatically, it is envisioned that the lactoferrin composition can reduce any of the following: the levels of circulating bacteria, the risk of the subject developing sepsis, septic shock, organ failure, and decrease the morbidity and mortality associated with bacteremia.

In a preferred embodiment of the present invention, the composition is administered in an effective amount to decrease, reduce, inhibit or abrogate the risk of developing bacteremia and minimizing the effects of already existing bacteremia, sepsis, septic shock or related conditions. The amount of lactoferrin in the composition may vary from about 1 mg to about 100 g. Preferably, the composition that is orally administered contains the range of 10 mg to 10 g of lactoferrin per day. More preferably, the composition contains the range of 1 mg to 50 g of lactoferrin per day. More preferably, the composition of the present invention also contains metal chelators, for example, but not limited to ethylenediaminetetraacetic acid (EDTA), [ethylene-bis-(oxyethylenenitrilo)]tetraacetic acid (EGTA), 1,2-bis-(2-aminophenoxy)ethane-N,N,N′,N′-tetraacetic acid (BAPTA), hydroxyethylethylene diamine triacetic acid, (HEDTA) or salts thereof. The amount of the metal chelator in the composition may vary from about 0.01 μg to about 20 g. A preferred metal chelator is EDTA. More preferably, the composition that is orally administered contains the ratio of 1:10,000 to about 2:1 EDTA to lactoferrin.

Treatment regimens may vary as well, and depend on the stage of bacterial infection and its consequences. The clinician will be best suited to make decisions on the best regimen to use based on the positive determination of the existing bacterial infection, the use of antibiotics and the known efficacy and toxicity (if any) of the therapeutic formulations. The guiding principle in the use of rhLF is to administer the treatment at the earliest signs of bacteremia, sepsis or septic shock being developed to attenuate the development of bacteremia and to reduce the extent of organ damage that results from sepsis and septic shock.

The improvement is any observable or measurable improvement. Thus, one of skill in the art realizes that a treatment may improve the patient or subject's condition, but may not be a complete cure of the disease. In certain aspects, the composition is administered in an effective amount to decrease, reduce, inhibit or abrogate levels of bacteria in circulation. In further aspects, an improvement can consist of any of the following, for example, decrease in the levels of circulating bacteria, attenuating the development of sepsis, attenuating the development of septic shock, attenuating the development of organ failure, decreasing morbidity associated with bacteremia and decreasing mortality (death) associated with bacteremia. Thus, after administration of lactoferrin, if any of the above conditions improve, then the amount of lactoferrin is considered to be an effective amount. Yet further, administration of lactoferrin will also attenuate the development of sepsis, septic shock and other conditions related thereto.

In certain aspects, the composition is administered in an effective amount to decrease, reduce, inhibit or abrogate the severity of sepsis or septic shock. In further aspects, an improvement can consist of any of the following, for example, decreasing mortality, decreasing morbidity, attenuating the development organ failure, decreasing days of hospitalization, decreasing or eliminating days of intensive care such as in an intensive care unit, decreasing or eliminating the use of supportive care such as a mechanical ventilator or decreasing the incidence of sequelae such as ARDS. Survival in patients with organ failure at baseline and prevention and reversal of organ failure are also evaluated. Thus, after administration of lactoferrin, if any of the above conditions improve, then the amount of lactoferrin is considered to be an effective amount.

In certain aspects, the composition is administered in an effective amount to decrease, reduce, inhibit or abrogate the severity of ALI or ARDS. In further aspects, an improvement can consist of any of the following, for example, decrease in mortality, attenuating the development organ failure, decreasing days of hospitalization, decreasing or eliminating days of intensive care such as in an intensive care unit, or decreasing or eliminating the use of supportive care such as a mechanical ventilator or PaO₂/FiO₂ ratios. Thus, after administration of lactoferrin, if any of the above conditions improve, then the amount of lactoferrin is considered to be an effective amount.

In specific embodiments, the composition is given in a single dose or multiple doses. The single dose may be administered daily, or multiple times a day, or multiple times a week. In a further embodiment, the lactoferrin is given in a series of doses. The series of doses may be administered daily, or multiple times a day, weekly, or multiple times a week. In a further embodiment, the lactoferrin is given as a continuous infusion via a nasogastric tube.

A further embodiment of the present invention is a method of treating bacteremia, sepsis, septic shock, related conditions or their consequences comprising the step of supplementing a mucosal immune system by increasing the amount of lactoferrin in the gastrointestinal tract. Preferably, the lactoferrin is administered orally.

Still yet, a further embodiment is a method of enhancing a mucosal immune response in the gastrointestinal tract in a subject comprising the step of administering orally to said subject the composition of the present invention. The composition contains lactoferrin alone or in combination with a metal chelator, such as EDTA. It is envisioned that the immune response is enhanced by lactoferrin stimulating cytokines and/or chemokines. Exemplary cytokines include interleukin-18 and GM-CSF in the gastrointestinal tract, which are known to enhance immune cells or stimulate production of immune cells. For example, interleukin-18 enhances natural killer cells or T lymphocytes. In specific embodiments, interleukin-18 (IL-18) enhances CD4+, CD8+ and CD3+ cells. It is known by those of skill in the art that IL-18 is a Th1 cytokine that acts in synergy with interleukin-12 and interleukin-2 in the stimulation of lymphocyte IFN-gamma production. Other cytokines or chemokines may also be enhanced for example, but not limited to IL-12, IL-1b, MIP-3α, MIP-1α or IFN-γ. Other cytokines or enzymes may be inhibited for example, but not limited to IL-2, IL-4, IL-5, IL-6, IL-10, TNF-α, or matrix metalloproteinases. It is further contemplated that lactoferrin inhibits the production of TNF-α, which inhibits cells involved in inflammation. It is also envisioned that lactoferrin stimulates interleukin-18 and a Th1 response following oral administration, which inhibits pro-inflammatory cytokines, e.g., IL-4, IL-5, IL-6, IL-8 and TNF-α.

The lactoferrin composition of the present invention can also result in inhibition of a cytokine or chemokine. The cytokines include, but are not limited to interleukin-2 (IL-2), interleukin-4 (IL-4), interleukin-5 (IL-5), interleukin-10 (IL-10), and tumor necrosis factor alpha (TNF-α). Still further, the lactoferrin composition can also inhibit the production of matrix metalloproteinases (MMPs).

In further embodiments, cytokines, for example, interleukin-18 or granulocyte/macrophage colony-stimulating factor, can stimulate the production or activity of immune cells. The immune cells include, but are not limited to T lymphocytes, natural killer cells, NK-T cells, macrophages, dendritic cells, and polymorphonuclear cells. More specifically, the polymorphonuclear cells are neutrophils and the T lymphocytes are selected from the group consisting of CD4+, CD8+ and CD3+ T cells.

Yet further, it is envisioned that oral administration of lactoferrin in combination with a metal chelator, such as EDTA, enhances the amount of metal ion that is sequestered and therefore enhances the effectiveness of lactoferrin in enhancing the immune system.

### D. COMBINATION TREATMENTS

In order to increase the effectiveness of the composition, it may be desirable to combine these compositions and methods of the invention with a known agent effective in the treatment or prevention of bacteremia, sepsis, septic shock and related conditions, for example known agents to treat bacterial infections, e.g., antibiotics, known agents for the treatment of sepsis, e.g., Drotrecogin alfa (activated) and agents to treat inflammation. In some embodiments, it is contemplated that a conventional therapy or agent, including but not limited to, a pharmacological therapeutic agent may be combined with the composition of the present invention.

The composition of the present invention may precede, be co-current with and/or follow the other agent(s) by intervals ranging from minutes to weeks. In embodiments where the composition of the present invention, and other agent(s) are applied separately to a cell, tissue or organism, one would generally ensure that a significant period of time did not expire between the time of each delivery, such that the composition and agent(s) would still be able to exert an advantageously combined effect on the cell, tissue or organism.

Various combination regimens of the composition and one or more agents are employed. One of skill in the art is aware that the composition of the present invention and agents can be administered in any order or combination. In other aspects, one or more agents may be administered substantially simultaneously, or within about minutes to hours to days to weeks and any range derivable therein, prior to and/or after administering the composition.

Administration of the composition to a cell, tissue or organism may follow general protocols for the administration of cardiovascular therapeutics, taking into account the toxicity, if any. It is expected that the treatment cycles would be repeated as necessary. In particular embodiments, it is contemplated that various additional agents may be applied in any combination with the present invention.

Pharmacological therapeutic agents and methods of administration, dosages, etc. are well known to those of skill in the art (see for example, the “Physicians Desk Reference”, Goodman & Gilman's “The Pharmacological Basis of Therapeutics”, “Remington's Pharmaceutical Sciences”, and “The Merck Index, Eleventh Edition”, incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject, and such individual determinations are within the skill of those of ordinary skill in the art.

Non-limiting examples of a pharmacological therapeutic agent that may be used in the present invention include an antimicrobial agent, an anti-sepsis agent, an anti-inflammatory agent, an antithrombotic/fibrinolytic agent, a blood coagulant, an antiarrhythmic agent, an antihypertensive agent, a vasopressor, or agents to treat metabolic acidosis. In certain aspects of the present invention, antimicrobial agents, e.g., antibiotics are used in combination with the composition of the present invention. Examples of specific antibiotics that can be used include, but are not limited to, erythromycin, nafcillin, cefazolin, imipenem, aztreonam, gentamicin, sulfamethoxazole, vancomycin, ciprofloxacin, trimethoprim, rifampin, metronidazole, clindamycin, teicoplanin, mupirocin, azithromycin, clarithromycin, ofloxacin, lomefloxacin, norfloxacin, nalidixic acid, sparfloxacin, pefloxacin, amifloxacin, gatifloxacin, moxifloxacin, gemifloxacin, enoxacin, fleroxacin, minocycline, linezolid, temafloxacin, tosufloxacin, clinafloxacin, sulbactam, clavulanic acid, amphotericin B, fluconazole, itraconazole, ketoconazole, and nystatin. Other examples of antibiotics, such as those listed in Sakamoto et al, U.S. Pat. No. 4,642,104 herein incorporated by reference will readily suggest themselves to those of ordinary skill in the art. Anti-sepsis agents include, but are not limited to Drotrecogin alfa (activated). Agents used for the treatment of ALI and ARDS include but are not limited to intra-pulmonary instillation of surfactants, and leukotriene modifiers. Anti-inflammatory agents include, but are not limited to non-sterodial anti-inflammatory agents (e.g., naproxen, ibuprofen, celeoxib) and sterodial anti-inflammatory agents (e.g., glucocorticoids).

Non-limiting examples of non-pharmacologic interventions that may be used in the present invention include supportive care such as organ support in sepsis and septic shock and low tidal volume ventilation protocols in ALI and ARDS.

### E. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Lipopolysaccharide-Induced Septic Shock in Mice—Model Characterization

In this experiment, the relationship between the dose of LPS and the mortality of test animals was determined. Groups of 10 C57BL/6J mice 18±1 g were used. Animals received different doses of E. coli lipopolysaccharide (LPS, 30, 20, 15 and 10 ng/mouse IV) and Vehicle (Saline, 0.2 ml/mouse) immediately after pre-treatment with D(+)-Galactosamine (20 mg/mouse IV). Mortality was recorded every 12 hours over a 3-day period. Table 2 illustrates that 20-30 ng/mouse of LPS resulted in 100% mortality and 15 ng/mouse of LPS resulted in 50% mortality.

TABLE 2
Murine Model of LPS Induced Sepsis

| | | | | Number of Death Recorded Every 12 Hours | | | | | | | % |
|---|---|---|---|---|---|---|---|---|---|---|---|
| Treatment | Route | Dose | N | 0-12 | 12-24 | 24-36 | 36-48 | 48-60 | 60-72 | Total | Mortality |
| Vehicle | IV | 0.2 ml/mouse | 10 | 0 | 0 | 0 | 0 | 0 | 0 | 0 | 0 |
| (saline) | | | | | | | | | | | |
| LPS | IV | 30 ng/mouse | 10 | 10 | 0 | 0 | 0 | 0 | 0 | 10 | 100 |
| LPS | IV | 20 ng/mouse | 10 | 8 | 2 | 0 | 0 | 0 | 0 | 10 | 100 |
| LPS | IV | 15 ng/mouse | 10 | 1 | 2 | 2 | 0 | 0 | 0 | 6 | 50 |
| LPS | IV | 10 ng/mouse | 10 | 0 | 1 | 1 | 0 | 0 | 0 | 2 | 20 |

### Example 2

### Effect of Intravenously Administered rhLF in an Murine LPS Model of Sepsis

Vehicle and test substance (rhLF) were administered intravenously to groups of 8 C57BL/6J male mice weighing 18 to 20 g, 60 minutes before and 10 minutes after challenge with lipopolysaccharide (LPS, from E. coli, LD100 of 20 ng/animal i.v.) plus galactosamine (20 mg/animal, i.v.). RhLF reduced the mortality induced by LPS by 38% shown in Table 3.

TABLE 3
Efficacy of IV rhLF in LPS-Induced Sepsis

| Treatment | Route | Dose | N | Deaths | Protection |
|---|---|---|---|---|---|
| Vehicle | IV | 0.2 ml/mouse × 2 | 8 | 8 | — |
| RhLF | IV | 500 μg/mouse × 2 | 8 | 5 | 38% |

### Example 3

### Effect of Orally Administered rhLF in an Murine LPS Model of Sepsis

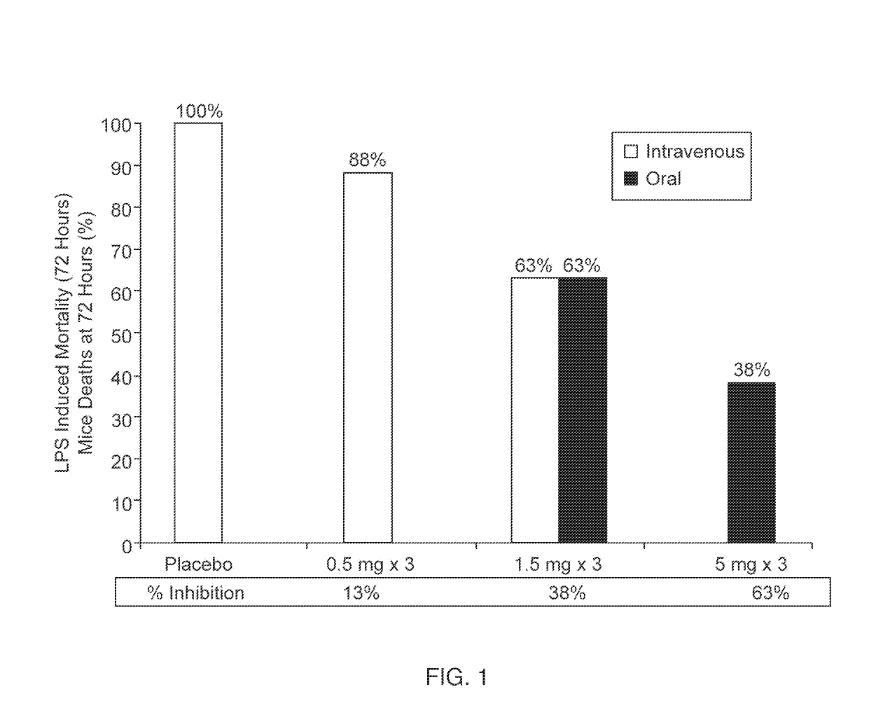

Recombinant human lactoferrin was administered orally and intravenously at doses of 5 and 1.5 mg/mouse (PO) and 1.5 and 0.5 mg/mouse (IV) at 1, 6 and 12 hours after challenge with Lipopolysaccharide plus galactosamine. FIG. 1 illustrated that orally administered rhLF provided a protection comparable to that provided by IV rhLF.

### Example 4

### Dose Dependent Protection of Oral rhLF in LPS-Induced Septic Shock

LPS and galactosamine were used to induce septic shock in four groups of ten mice each as described in Example 1. The mouse cohorts then received either placebo or one of three doses of rhLF (1.5 mg, 5 mg or 10 mg per dose) administered by oral gavage at 1 hour, 6 hours and 12 hours after LPS administration. Oral rhLF provided a dose dependent protection against LPS induced mortality as shown in Table 4.

TABLE 4
RhLF provides a dose dependent protection in LPS-induced sepsis

| Treatment | Dose | N | Deaths | Protection |
|---|---|---|---|---|
| Vehicle | — | 10 | 10 | — |
| RhLF | 1.5 mg/dose | 10 | 6 | 40% |
| RhLF | 5 mg/dose | 10 | 5 | 50% |
| RhLF | 10 mg/dose | 10 | 4 | 60% |

### Example 5

### Efficacy of Oral rhLF Administered by Different Regimens in LPS-Induced Septic Shock

LPS and galactosamine were used to induce septic shock in five groups of 8-10 mice each as described in Example 1. The mouse cohorts received either placebo or 5 mg/dose of rhLF administered by oral gavage in one of four different regimens. Oral rhLF provided protection against LPS induced mortality when administered either prophylactically or therapeutically and in all the regimens tested as shown in Table 5.

TABLE 5
RhLF provides protection in LPS-induced sepsis used in
different regimens

| Treatment | Regimen* | N | Deaths | Protection |
|---|---|---|---|---|
| Vehicle | +1, +6, +12 | 10 | 10 | — |
| RhLF | +1, +6, +12 | 10 | 5 | 50% |
| RhLF | −1, +1, +6, +12 | 8 | 3 | 63% |
| RhLF | −+1, +12 | 8 | 6 | 25% |
| RhLF | −+1, +4, +8, +12 | 8 | 4 | 50% |
| RhLF | +6, +12 | 8 | 6 | 25% |

*Placebo/RhLF administration in hours relative to the LPS dose

### Example 6

### Protective Effect of rhLF in a Murine Model of Bacteremia

Groups of 10 ICR derived male or female mice weighing 22±2 g were used. Each animal was inoculated intraperitoneally with E. coli (ATCC 25922; 1−3×10⁵ CFU/mouse) suspended in 0.5 ml brain-heart infusion broth containing 5% mucin.

Vehicle or rhLF (10 mg/mouse), was administered 1, 6, 12, and 24 hours following the E. coli administration and mortality was measured over 7 days. RhLF treated animals showed a 10% reduction in mortality relative to the placebo treated animals as shown in Table 6.

TABLE 6
Protective effect of rhLF

| Treatment | Dose | N | Death | Protection |
|---|---|---|---|---|
| Vehicle | 5 mL/kg × 6 | 10 | 10 | 0% |
| RhLF | 10 mg/mouse | 10 | 9 | 10% |

### Example 7

### Protective Effect of rhLF in a Sublethal Murine Model of Bacteremia

Groups of 10 ICR derived male or female mice weighing 22±2 g are used. Each animal is inoculated intraperitoneally with a sub-lethal dose of E. coli (ATCC 25922) suspended in 0.5 ml brain-heart infusion broth containing 5% mucin.

Test substance, rhLF or vehicle, are administered using the following doses and administration schedules: Gp. 1-10 mg/mouse @ 1, 6, 12 and 24 hours after bacterial inoculation; Gp. 2-10 mg/mouse @ 1, 6, 12, 24, 48 and 72 hours after bacterial inoculation; Gp. 3—vehicle control, administered @ 1, 6, 12, 24, 48 and 72 hours after bacterial inoculation; N=10 group; total N=30 animals. Mortality is recorded daily during the following 7 days.

### Example 8

### Protective Effect of rhLF in Combination with an Antibiotic in a Sublethal Murine Model of Bacteremia

Groups of 10 ICR derived male or female mice weighing 22±2 g are used. Each animal is inoculated intraperitoneally with a sub-lethal dose of E. coli (ATCC 25922) suspended in 0.5 ml brain-heart infusion broth containing 5% mucin. Animals are treated with kanamycin antibiotic at a dose of 100 mg/kg/day.

Test substance, rhLF or vehicle, are administered using the following doses and administration schedules: Gp. 1-10 mg/mouse @ 1, 6, 12 and 24 hours after bacterial inoculation; Gp. 2-10 mg/mouse @ 1, 6, 12, 24, 48 and 72 hours after bacterial inoculation; Gp. 3—vehicle control, administered @ 1, 6, 12, 24, 48 and 72 hours after bacterial inoculation; N=10 group; total N=30 animals. Mortality is recorded daily during the following 7 days.

### Example 9

### Protective Effect of Oral rhLF Administration in a Baboon Model of Sepsis

In a prospective, randomized, placebo-controlled study, the efficacy of oral rhLF is studied over 72 hours in chronically instrumented male baboons infused with live E. coli under antibiotic therapy.

Recombinant human lactoferrin is administered prophylactically at the time of administration of antibiotics. Baboons randomly receive either placebo or are administered rhLF orally at doses of 100, 200 or 400 mg/kg/day.

The primary outcome measure is the mortality rate. Major morbidities are considered as secondary end points. Safety of rhLF administration is also monitored.

The pharmacological effects of the treatment are evaluated by measuring changes in the concentration of important cytokines, namely IL-18, IL-1, IL-2, IL-4, IL-5, IL-8, IL-10, IL-12, IFN-gamma and TNF-alpha in the serum collected at the various time points of treatment.

### Example 10

### Protective Effect of rhLF in Bacteremia, Sepsis and Septic Shock when Co-Administered with EDTA

Recombinant human lactoferrin is co-administered with EDTA, at a weight ratio of 1:1, orally or intravenously at doses of 5 and 1.5 mg/mouse of rhLF and 5 and 1.5 mg/mouse of EDTA, respectively (PO) and 1.5 and 0.5 mg/mouse and 1.5 and 0.5 mg/mouse of EDTA, respectively (IV) at 1, 6 and 12 hours after challenge with Lipopolysaccharide plus galactosamine. Mortality is recorded over a 3-day period. Reduction of mortality by 50 percent or more 50%) relative to vehicle treated group indicates significant protection.

### Example 11

### Clinical Study on the Safety of rhLF Administered to Treat Bacteremia and Sepsis

Patients are selected for the study, based on the presence of symptoms of bacteremia—fever>101° F. (38.3° C.), chills, malaise, abdominal pain, nausea, vomiting, diarrhea, anxiety, shortness of breath and confusion. Most likely bacterial infections are due to staphylococcus, pseudomonas, haemophilus and E. coli. The subsequent septic shock usually occurs in immunocompromised or chronically ill patients.

Diagnosis of sepsis is made on the presence of at least two out of the following criteria: tachycardia (heart rate>90 bpm), hyperventilation (respiratory frequency>20/min or pCO₂exp<35 mm Hg), fever (>38° C.) or hypothermia (<36° C.), and leukocytosis (>12,000/μL) or leukopenia (<4,000/μL).

Recombinant human lactoferrin is administered prophylactically at the time of administration of antibiotics to treat the underlying infection, or at any other later, earliest possible time when the symptoms are present. Patients randomly receive either placebo or an escalating dose of rhLF administered orally at doses of 100, 200 or 400 mg/kg/day for 3 days. The protocol starts with the lowest dose and the safety of administration is evaluated before progressing to the next dose. For patients not able to take a p.o. dose, an infusion for 96 hours at the same dose level using a nasogastric tube is employed.

The primary outcome of this study is the evaluation of safety of rhLF administered to patients with bacteremia and sepsis.

### Example 12

### Clinical Study Showing the Protective Effect of rhLF in Sepsis—Dose Ranging

Patients are selected for the study, based on the presence of symptoms of bacteremia—fever>101° F. (38.3° C.), chills, malaise, abdominal pain, nausea, vomiting, diarrhea, anxiety, shortness of breath and confusion. Most likely bacterial infections are due to staphylococcus, pseudomonas, haemophilus and E. coli. The subsequent septic shock usually occurs in immunocompromised or chronically ill patients.

Diagnosis of sepsis is made on the presence of at least two out of the following criteria: tachycardia (heart rate>90 bpm), hyperventilation (respiratory frequency>20/min or pCO₂exp<35 mm Hg), fever (>38° C.) or hypothermia (<36° C.), and leukocytosis (>12,000/μL) or leukopenia (<4,000/μL).

Recombinant human lactoferrin is administered prophylactically at the time of administration of antibiotics to treat the underlying infection, or at any other later, earliest possible time when the symptoms are present. Patients randomly receive either placebo or are administered rhLF orally at doses of 100, 200 or 400 mg/kg/day. For patients not able to take a p.o. dose, a nasogastric infusion for 96 hours at the same dose level is used.

The primary outcome measure is the mortality rate. Major morbidities are considered as secondary end points. Safety of rhLF administration is also monitored. The patients are followed for at least 90 days.

The pharmacological effects of the treatment are evaluated by measuring changes in the concentration of important cytokines, namely IL-18, IL-1, IL-2, IL-4, IL-5, IL-8, IL-10, IL-12, IFN-gamma and TNF-alpha in the serum collected at the various time points of treatment.

### Example 13

### Clinical Study Showing the Protective Effect of rhLF in Bacteremia and Sepsis Phase 3 Study

Patients are selected for the study, based on the presence of symptoms of bacteremia—fever>101° F. (38.3° C.), chills, malaise, abdominal pain, nausea, vomiting, diarrhea, anxiety, shortness of breath and confusion. Most likely bacterial infections are due to staphylococcus, pseudomonas, haemophilus and E. coli. The subsequent septic shock usually occurs in immunocompromised or chronically ill patients.

Diagnosis of sepsis is made on the presence of at least two out of the following criteria: tachycardia (heart rate>90 bpm), hyperventilation (respiratory frequency>20/min or pCO₂exp<35 mm Hg), fever (>38° C.) or hypothermia (<36° C.), and leukocytosis (>12,000 μL) or leukopenia (<4,000/μL).

In this randomized, double-blind, multi-dose, placebo-controlled multicenter study, recombinant human lactoferrin is administered to ICU patients with symptoms of bacteremia and/or sepsis. Patients randomly receive either placebo or are administered rhLF orally at a rate of 400 mg/kg/day. For patients not able to take a p.o. dose, a nasogastric infusion for 96 hours at the same dose level is used.

The primary outcome measure is the all-cause 28-day mortality rate for treated patients. Secondary efficacy variables include time to death during 28-day follow up, and the patient status at time of discharge (up to 90 days). Major morbidities are considered as secondary end points (Multiple Organ Dysfunction Scores (MODS) and Sepsis-Related Organ Failure Assessment (SOFA). Survival in patients with organ failure at baseline and prevention and reversal of organ failure are also evaluated. The patients are followed for at least 90 days.

### Example 14

### Clinical Study Showing the Protective Effect of rhLF/XiGris® Combination in Bacteremia and Sepsis—Phase 2 Study

Patients are selected for the study, based on the presence of symptoms of bacteremia—fever>101° F. (38.3° C.), chills, malaise, abdominal pain, nausea, vomiting, diarrhea, anxiety, shortness of breath and confusion. Most likely bacterial infections are due to staphylococcus, pseudomonas, haemophilus and E. coli. The subsequent septic shock usually occurs in immunocompromised or chronically ill patients.

Diagnosis of sepsis is made on the presence of at least two out of the following criteria: tachycardia (heart rate>90 bpm), hyperventilation (respiratory frequency>20/min or pCO₂exp<35 mm Hg), fever (>38° C.) or hypothermia (<36° C.), and leukocytosis (>12,000 μL) or leukopenia (<4,000/μL).

In this randomized, double-blind, multi-dose, placebo-controlled multicenter study, recombinant human lactoferrin is administered to ICU patients with symptoms of bacteremia and/or sepsis. Patients randomly receive either Xigris® (Drotrecogin alfa, activated) @ 24 μg/kg/h infused over 96 hours or are administered rhLF orally at a rate of 400 mg/kg/day together with Xigris® (Drotrecogin alfa, activated) infusion @ 24 μg/kg/h infused over 96 hours. For patients not able to take rhLF dose as a p.o. dose, an nasogastric infusion for 96 hours at the same dose level is used.

The primary outcome measure is the all-cause 28-day mortality rate for treated patients. Secondary efficacy variables include time to death during 28-day follow up, and the patient status at time of discharge (up to 90 days). Major morbidities are considered as secondary end points (Multiple Organ Dysfunction Scores (MODS) and Sepsis-Related Organ Failure Assessment (SOFA). Survival in patients with organ failure at baseline and prevention and reversal of organ failure are also evaluated. The patients are followed for at least 90 days.

### Example 15

### Clinical Study Showing the Protective Effect of rhLF in ARDS—Phase 2 Study

In this randomized, controlled study, each patient is randomized to either the 12 mL/kg or 6 mL/kg ventilation treatment group and between rhLF or placebo. The rhLF arm is placebo-controlled and double-blinded.

RhLF has anti-inflammatory and immunomodulatory properties, with previous studies suggesting efficacy in ARDS prevention. This study is designed to test whether the oral administration of rhLF early after the onset of acute lung injury or ARDS will reduce mortality and morbidity.

Patients randomly receive either placebo or are administered rhLF orally at a rate of 400 mg/kg/day.

For patients not able to take a p.o. dose, an nasogastric infusion for 96 hours at the same dose level is used.

The primary outcome measure is the mortality rate. Major morbidities are considered as secondary end points. The patients are followed for at least 90 days.

### Example 16

### Reduction of Mortality and Key Cytokines in Sepsis

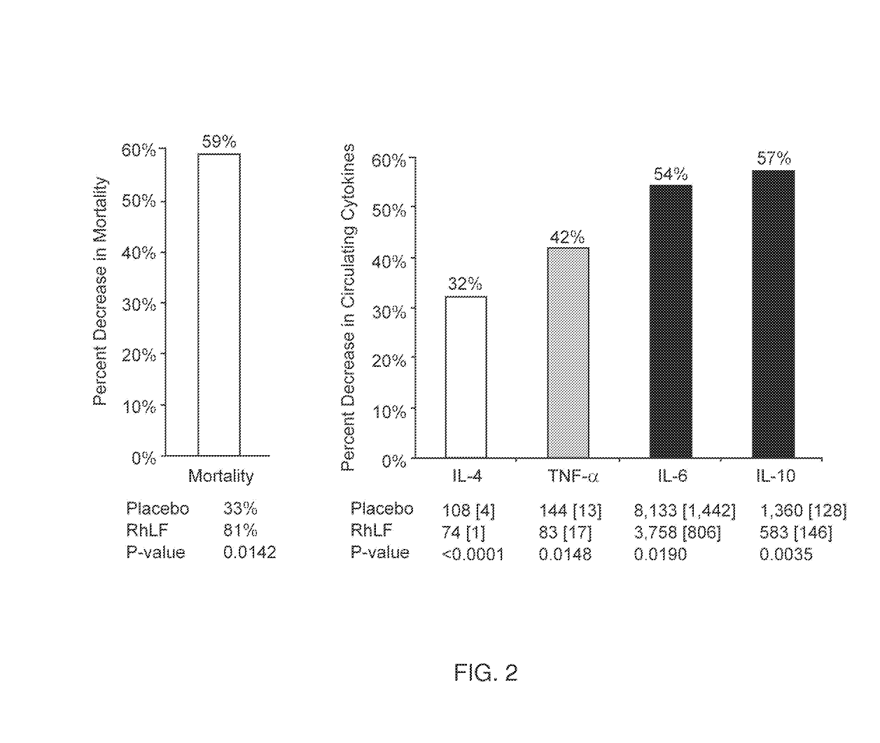

The protective effect of oral rhLF in LPS-induced sepsis was correlated with known cytokine modulators. Mice were treated with LPS (20 ng IV)+galactosamine. Animals received either placebo (−1, +1, +6, +12 Hours) or rhLF (5 mg/dose) by oral gavage relative to the LPS challenge. Mortality was measured out to 72 hours though all deaths occurred within 24 hours. Circulating cytokines were measured by ELISA 45 minutes after LPS challenge. Cytokine cohorts were four animals per group. A total of 28 animals (12 placebo, 16 rhLF) were followed for survival. 1-tailed p-values compared the placebo and rhLF animals. Values for individual cytokines are shown as averages. Consistent with earlier experiment, rhLF treated animals showed a significant 59% reduction in mortality. In addition, a reduction in circulating levels of IL-4, IL-6 and IL-10 was observed (FIG. 2), all Th2 cytokines previously reported to play a role in the pathophysiology of sepsis.

### Example 17

### Anti-Sepsis Activity of L005 Versus L006

The biological activity of three batches of rhLF that differed in the percentage of N-1 truncates was compared using a mouse model of LPS-induced sepsis. Placebo or rhLF (5 mg/dose) was administered orally to groups of 10 C57BL/6J male mice weighing 18 to 20 g at 1, 6 and 12 hours after challenge with LPS (20 ng/animal IV) plus galactosamine (20 mg/animal IV). Mortality was monitored every 12 hours over a period of 72 hours. As shown in Table 7, different batches of rhLF, with widely varying concentrations of the N-1 truncate, all provided similar protection against LPS induced mortality.

TABLE 7
Anti-Sepsis Effect of Three Different RhLF Preparations

| RhLF Batch | Placebo | L005 | L006 | D7001 |
|---|---|---|---|---|
| Number of Animals | 10 | 10 | 10 | 10 |
| Number of Deaths | 10 | 6 | 5 | 5 |
| % of N-1 Truncate | N/A | 27% | 50% | 0 |
| % Protection | N/A | 40% | 50% | 50 |

### Example 18

### Lack of Bioavailability of Oral rhLF in Mice

Custom-synthesized ¹⁴C-labeled rhLF (Perkin-Elmer Life Sciences) was administered orally to CD-1 mice to determine the extent of the protein absorption. Mice were inoculated with ¹⁴C-rhLF, and blood and tissue samples were collected as indicated in the Table 8 below.

TABLE 8
Doses of ¹⁴C-labeled rhLF administered to mice

| | | | Sample collection |
|---|---|---|---|
| | # of mice | dose | time at [min] |
| Gp 1 | 2 | 2 μCi (16.8 mg/kg) | 15 |
| Gp 2 | 2 | 8 μCi (67.2 mg/kg) | 15 |
| Gp 3 | 2 | 8 μCi (67.2 mg/kg) | 30 |

At the stated time, the mice were euthanized and blood and tissues were collected for analyses. Tissues were homogenized in a buffer containing protease inhibitor to prevent protein degradation. Blood was processed to plasma. Samples of plasma and tissue homogenates were counted on a scintillation counter, and also run on a PAGE chromatography to separate sample components by size. The gel contents were blotted onto a membrane that was exposed to a phosphorus imaging screen to detect ¹⁴C-labeled bands. The screen was capable of detecting as few as 500 cpm. The values of recorded counts are shown in the Table 9 below.

TABLE 9
Distribution of ¹⁴C-labeled rhLF after oral administration
Total CPM per organ (as % of inoculum)

| Treatment | Plasma | Blood cells | Liver | Kidneys | Lungs | Spleen |
|---|---|---|---|---|---|---|
| Gp 1 | 0.94 | 0.076 | 3.06 | 0.5 | 0.09 | 0.038 |
| | 1.06 | 0.095 | 6.02 | 0.37 | 0.11 | 0.046 |
| Gp 2 | 0.55 | 0.024 | 10.4 | 0.93 | 0.21 | 0.089 |
| | 0.29 | 0.015 | 5.4 | 0.42 | 0.1 | 0.028 |
| Gp 3 | 1.11 | 0.030 | 9.08 | 0.9 | 0.2 | 0.088 |
| | 1.36 | 0.028 | 8.19 | 1.24 | 0.19 | 0.249 |

While counts were found in plasma and tissues, this is consistent with the known degradation of rhLF in the stomach and small intestine after oral administration. There was no detectable intact rhLF seen on PAGE gels, confirming that, within the limits of detection, no rhLF protein is absorbed after oral administration.

### Example 19

### Lack of Bioavailability of Oral rhLF in Humans

Recombinant human lactoferrin was administered orally to five separate groups of healthy human subjects. These five cohorts were randomized at 6:1 ratio for rhLF and placebo (using 35 subjects in total). The doses are shown in the Table 10 below.

TABLE 10
Doses and dose regimens administered to human subjects

| Group | G1 | G2 | G3 | G4 | G5 |
|---|---|---|---|---|---|
| Dose | 4.5 g once | 0.5 g × 7 days | 1.5 g × 7 days | 1.5 g TID × 7 days | 4.5 g BID × 7 days |

Blood samples were collected from 0 to 48 hours for Group 1, or over the dosing interval on Days 1 and 7 for Groups 2 and 5. Plasma LF was determined using a validated ELISA method. The values of LF concentration showed high inter-subject variability for both rhLF— and placebo-treated subjects. Since the ELISA determines both the endogenous and recombinant human lactoferrin, the extent of absorption of rhLF after oral administration could only be estimated against the inter-subject variations in endogenous LF production and turnover. No relationship between the blood levels of LF and the administered dose of rhLF was apparent. There was no measurable increase in LF levels on Day 7 of repeated daily administration. Based on these data, the oral bioavailability of rhLF was estimated to be <0.5%. (Mojaverian P et al.)

### REFERENCES CITED

All patents and publications mentioned in the specifications are indicative of the levels of those skilled in the art to which the invention pertains. All patents and publications are herein incorporated by reference to the same extent as if each individual publication was specifically and individually indicated to be incorporated by reference.
U.S. Pat. No. 5,571,691
U.S. Pat. No. 5,571,697
U.S. Pat. No. 5,571,896
U.S. Pat. No. 5,629,001
Kruzel M L, et al., Clin Exp Immunol 2002; 130:25-31
Heyman M and Desjeux J-F, J Pediatr Gastroenterol Nutr 1992; 15:48-57
Fransson G B, et al., Nutr Res 1983; 3:373-84
Holloway N M, et al., Am J Vet Res 2002 April; 63(4):476-8
Mojaverian P. et al. Proceedings of the Annual Meeting of the American Association of Pharmaceutical Scientists, 2003.

Although the present invention and its advantages have been described in detail, it should be understood that various changes, substitutions and alterations can be made herein without departing from the spirit and scope of the invention as defined by the appended description. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended descriptions are intended to include within their scope such processes, machines, manufacture, compositions of matter, means, methods, or steps.

What is claimed is:

1. A method of treating a septic shock in a human subject comprising the step of administering orally to said human subject an effective amount of a human lactoferrin composition to provide an improvement in the septic shock of said human subject.

2. The method of claim 1, wherein the improvement is a decrease in morbidity of said subject.

3. The method of claim 1, wherein the improvement is a decrease in mortality of said subject.

4. The method of claim 1, further comprising administering the lactoferrin composition in combination with an antibiotic.

5. The method of claim 1, further comprising administering the lactoferrin composition in combination with Drotrecogin alfa (activated).

6. The method of claim 1, wherein the septic shock in the human subject comprises an organ failure.

7. The method of claim 6, wherein the improvement comprises attenuating organ failure.
